(19) United States
(12) Patent Application Publication
(10) Pub. No.: US 2012/0006147 A1
(43) Pub. Date: Jan. 12, 2012

(54) OPERATOR CONDITION DETECTING DEVICE AND STEERING WHEEL

(75) Inventor: Satoshi SANO, Kawasaki (JP)

(73) Assignee: FUJITSU LIMITED, Kawasaki (JP)

(21) Appl. No.: 13/243,211

(22) Filed: Sep. 23, 2011

Related U.S. Application Data
(63) Continuation of application No. PCT/JP2009/058706, filed on May 8, 2009.

(51) Int. Cl. A61B 5/0408; A61B 5/0478; A61B 5/0492; B62D 1/04

(52) U.S. Cl. 74/552; 600/509

(57) ABSTRACT

When the electrical condition of a driver who is an operator of a vehicle is acquired by providing an electrode part on a steering wheel or the like that comes into contact with the driver, the electrode part is formed on a steering-wheel structure and is covered with a protective member. Conductive parts that pass through the protective part are formed by way of holes that are formed in the protective member. The conductive parts make the hand of the driver touching the steering wheel be in electrical contact with the electrode part. This configuration enables detection of the electrical condition of the operator without losing the degree of freedom in design by increasing the durability and easing the restrictions on the material and shape.

### CROSS-REFERENCE TO RELATED APPLICATION

This application is a continuation of International Application No. PCT/JP2009/058706, filed on May 8, 2009, the entire contents of which are incorporated herein by reference.

### FIELD

The embodiment discussed herein is directed to an operator condition detecting device that detects the electrical condition of an operator of an apparatus and is also directed to a steering wheel.

### BACKGROUND

An operator is needed to be in an appropriate psychological state when the operator operates an apparatus. For example, if an operator falls asleep or is careless when the operator operates a vehicle or an industrial machine, the operator may cause a serious accident.

It is known that electrocardiographic measurement is effective for picking up changes in the arousal level of an operator. It is known that, particularly, heart rate variability includes a sign of a decrease in arousal levels. Therefore, heart rate measurement is under consideration for the purpose of monitoring conditions, such as drowsiness, of an operator (driver) who operates (drives) an apparatus.

If the apparatus is, for example, a vehicle, detection of the heart rate is enabled by providing electrodes on the steering wheel of the vehicle and measuring the cardiac action potential between both hands via the electrodes. Conventionally, a technology has been disclosed in which the heart rate is measured by measuring the potential between both hands of a driver while driving a vehicle by using electrodes provided on the steering wheel of the vehicle.

Moreover, there have conventionally been proposed a biological information detecting apparatus that detects an electrocardiographic signal by using electrodes formed on a steering wheel, a contact member used therefor, and a paint for a biological information detecting member used therefor. To realize this configuration, there is further disclosed a designing method for setting the impedance between electrodes to 1/100 as a circuit condition for measurement and a method for deciding an impedance-based design condition and a material condition as a design condition of electrodes of a steering part.

Patent document 1: Japanese Laid-open Patent Publication No. 2002-102188

Patent document 2: International Publication Pamphlet No. WO2004/089209

However, if electrodes are formed on the surface of a steering part as in the case of the conventional technology, there is a problem in that the durability decreases depending on the steering actions because the electrodes are exposed on the steering part. Especially, if a crack occurs due to an impact and stress during the steering actions, there is the possibility that a contact-surface area is disconnected from a wiring part.

Even if a durable material is selected as the material for the electrodes, there is the possibility that a loss occurs in the grip performance and the steering performance because materials and shapes are limited.

### SUMMARY

According to an aspect of an embodiment of the invention, an operator condition detecting device includes an electrode part that detects an electrical condition of an operator of an apparatus, a protective layer that covers the electrode part, a plurality of holes that passes through the protective layer, and conductive parts that are formed on inner walls of the holes to be in contact with the electrode part.

The object and advantages of the embodiment will be realized and attained by means of the elements and combinations particularly pointed out in the claims.

It is to be understood that both the foregoing general description and the following detailed description are exemplary and explanatory and are not restrictive of the embodiment, as claimed.

### BRIEF DESCRIPTION OF DRAWINGS

FIG. 1 is a diagram of a configuration of an operator condition detecting device according to the present embodiment;

FIG. 2 is an explanatory diagram of conductive parts;

FIG. 3 is a diagram that explains detection of an electrical condition of a driver;

FIG. 4 is a diagram that illustrates an equivalent circuit including the driver himself/herself;

FIG. 5 is an explanatory diagram of an electrocardiogram waveform;

FIG. 6 is a diagram that explains peak cycle detection using the electrocardiogram waveform;

FIG. 7 is a structure diagram of a seat-surface electrode part that detects an electrocardiographic signal from the right buttock and the left buttock;

FIG. 8 is a diagram that explains a way of individually detecting electrical conditions from the right buttock and the left buttock of the driver;

FIG. 9 is a diagram that illustrates an equivalent circuit that is used to individually detect electrical conditions from the right buttock and the left buttock of the driver;

FIG. 10 is a diagram that explains a configuration in which electrodes are provided on the outside and the inside of a steering wheel;

FIG. 11 is a diagram of a configuration in which electrical conditions are detected from the outside and the inside of the steering wheel;

FIG. 12 is a diagram that explains a configuration in which electrodes are provided on not only the right side and the left side of the steering wheel but also the upper side of the steering wheel;

FIG. 13 is a diagram that explains a configuration in which auxiliary electrodes are provided on the steering wheel;

FIG. 14 is a diagram that explains the shape of holes formed on a protective member;

FIG. 15 is a diagram that explains an example in which the protective member is sewn with conducting thread;

FIG. 16 is a diagram that explains a configuration in which the entire steering wheel is covered with the protective member; and

FIG. 17 is a diagram that explains a configuration in which a conductive layer is further formed on the surface of the protective member.

### DESCRIPTION OF EMBODIMENT

A preferred embodiment of the present invention will be explained with reference to accompanying drawings. It is noted that the present invention is not limited to the following embodiment.

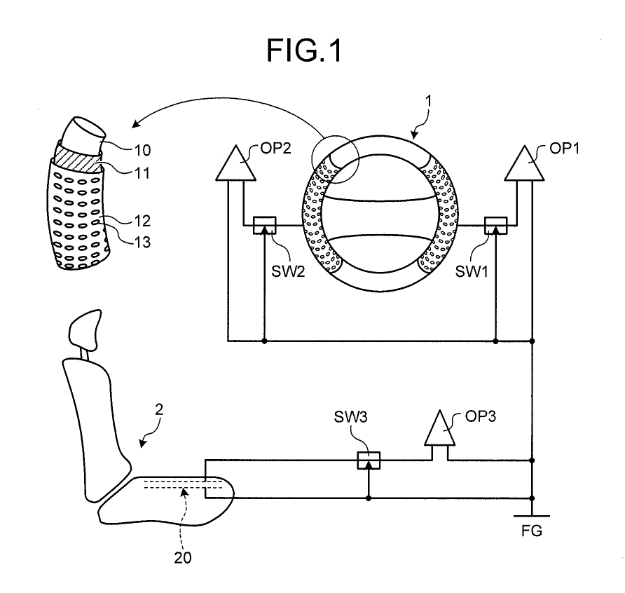

In the present embodiment, an example is explained where the disclosed technology is applied to a steering wheel and a driver seat of a vehicle. FIG. 1 is a diagram of a configuration of an operator condition detecting device according to the present embodiment. The operator condition detecting device detects the electrical conditions via a steering wheel 1 from the right and left hands of an operator, i.e., the driver of a vehicle. In the same manner, the operator condition detecting device detects the electrical condition from the buttocks of the driver by using a seat-surface electrode part 20 of a driver seat 2.

In the steering wheel 1, an electrode 11 that detects the electrical condition of the driver is formed on a steering-wheel structure 10. The electrode 11 is coated with a protective member 12. The protective member 12 has a plurality of holes 13 formed thereon. The holes 13 pass through the protective member 12. Conductive parts 14 described below are formed on the inner walls of the holes 13. The conductive parts 14 are in contact with the electrode 11. Therefore, when the driver grips the steering wheel 1, the hands of the driver are electrically in contact with the electrode 11 via the conductive parts 14.

The protective member 12 can be made of a nonconductive material, such as leather. Coating a steering wheel with leather or the like has been practiced conventionally. Forming many holes in a member that coats a steering wheel has also been practiced conventionally. Coating a steering wheel with a coating member and forming holes in the coating member have been practiced conventionally for the purpose of improvement in the grip performance of the steering wheel and, in turn, improvement in the steering performance. Moreover, coating a steering wheel with a coating member and forming holes in the coating member have been employed as a design.

Because the conductive parts 14 are provided on the inner walls of the holes 13 of the protective member 12, it is possible to acquire the electrical conditions from the hands of the driver while using the design of a steering wheel that is widely used. Moreover, even if the protective member 12 is worn away or damaged, the operator condition detecting device can still acquire the electrical condition of the driver.

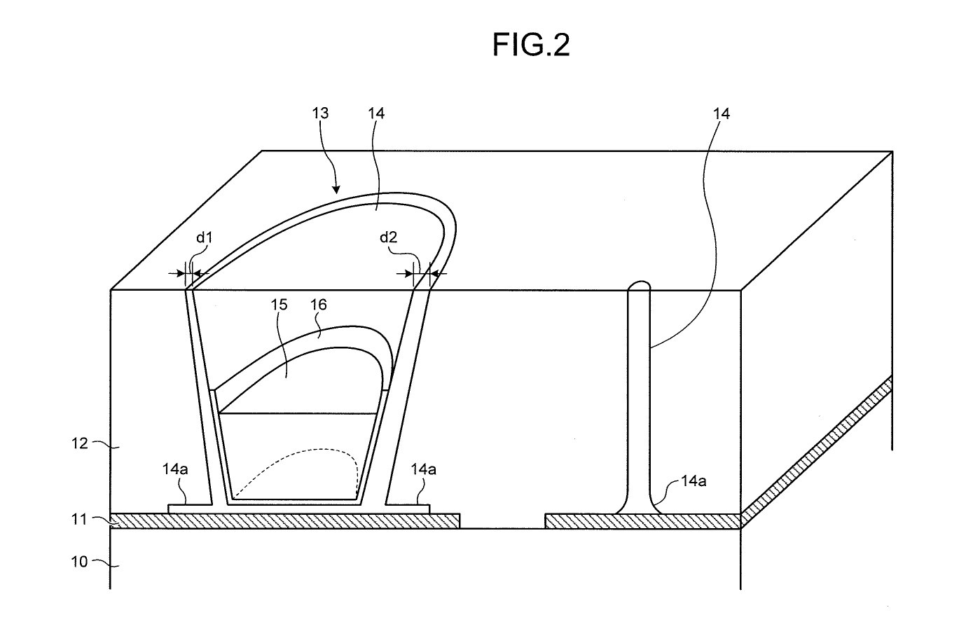

FIG. 2 is an explanatory diagram of conductive parts. As illustrated in FIG. 2, the layer-shaped electrode 11 is formed on the steering-wheel structure 10 and is coated with the protective member 12. Each of the holes 13 formed in the protective member 12 passes through the protective member 12 and has an electrode-side open end and a protective-member-surface-side open end. The electrode-side open end of the hole 13 is smaller than the protective-member-surface-side open end. Thus, the hole 13 is tapered.

The conductive part 14 is formed on the inner wall of the hole 13 and is in contact with the electrode 11. The conductive part 14 has a auxiliary contact surface 14a at a contact site between the conductive part 14 and the electrode 11. The auxiliary contact surface 14a increases the area where the electrode 11 is in contact with the conductive part 14, which improves the electric properties, for example, decreases the resistance.

It is preferable that the thickness of the conductive part 14 increases as it comes closer to the electrode 11 and the thickness decreases as it comes closer to the surface of the protective member 12. The material of the conductive part 14 is preferably a conductive material including metallic powder, such as nickel. In contrast, the material of the protective member 12 is in general a material, such as leather, cloth, and resin, softer than the material of the conductive part 14. The reason why a soft material is used as the protective member 12 is to make the touch of the steering wheel soft and to ensure its grip.

If the surface of the steering wheel 1 is worn away when the material of the protective member 12 is softer than the material of the conductive part 14, the protective member 12 is likely to be worn further away than the conductive part 14. If the difference between the abrasion rates is large, the conductive part 14 protrudes out of the worn-away protective member 12, and thus the steering performance may decrease.

If the thickness of the conductive part 14 decreases as it comes closer to the surface of the protective member 12, the conductive part 14 close to the surface of the protective member 12 is likely to be worn away. In other words, by decreasing the difference between the abrasion rates of the protective member 12 and the conductive part 14 near the surface of the protective member 12, the situation where the protective member 12 is first worn away and thus the conductive part 14 protrudes can be prevented.

It is unnecessary that the thickness of the conductive part 14 is uniform on the surface of the protective member 12. In FIG. 2, d1 and d2 are examples of thicknesses of the conductive part 14 on the surface-side of the protective member 12. If the steering wheel 1 is frequently operated to be worn away in a particular direction, the thicknesses d1 and d2 are decided in accordance with the direction. For example, if the steering wheel is frequently operated to be worn away in the direction from d1 to d2, d1 is set thinner than d2.

The open ends of the hole 13 can have an arbitrary shape, such as a circle and a rhombus. An example where the open ends of the hole 13 are elliptically shaped is illustrated in FIG. 2. Accordingly, the shape of the conductive part 14 also becomes an elliptical shape. Because the hole 13 and the conductive part 14 are shaped elliptically, the resistance to abrasion in the minor axis direction is higher than the resistance to abrasion in the major axis direction. When the configuration is applied to the steering wheel 1, it is preferable to align the major axis of the elliptical shape to the radial direction and the minor axis to the circumferential direction to increase the resistance to abrasion in the circumferential direction because the steering wheel is frequently operated in the outer circumferential direction and thus the outer-circumferential-direction abrasion is severe.

Moreover, the electrode-side inside of the conductive part 14 is coated with a coating 16 and then filled with a resin 15 that acts as a protective agent. The coating 16 and the resin 15 prevent deterioration and corrosion of the bottom part of the conductive part 14 and the electrode 11. Due to coating of the coating 16 and filling of the resin 15, the area where the conductive part 14 can be in electric contact with the hands of the driver is limited to an area from the surface of the protective member 12 to near the surface of the resin 15. In other words, when the driver grips the steering wheel 1, skin on the palms and the fingers comes inside the conductive part 14 to be in contact with the conductive part 14 but does not go deeper beyond the resin 15; therefore, the skin is not in contact with the bottom part of the conductive part 14 and the electrode 11.

Because the hole 13 has a slip-proof role of the steering wheel, it is preferable to decide the size and the layout of holes in such a manner that the holes are included in an area with which the palms and the fingers come into contact. If the hole 13 is small sufficiently, the palms and the fingers do not touch the electrode 11 and the bottom part of the conductive part 14 even if the holes are not coated with the coating 16 or filled with the resin 15.

More particularly, it is anticipated that the thickness of the protective member 12 is from 1 to 2 mm and the diameter of the hole 13 is from 1 to 2 mm.

Another configuration in which the bar-shaped or thread-shaped conductive part 14 passes through the protective member 12 without providing any opening is illustrated in FIG. 2. The bar-shaped conductive part 14 can be formed by forming a hole in the protective member 12 and then filling the hole with a conductive material. As another example of the forming method, it can be formed by driving a bar-shaped or rivet-shaped conductor into the protective member 12. The thread-shaped conductive part 14 can be formed by sewing the protective member 12 with conducting thread. It is noted that the diameter of the bar-shaped or the thread-shaped conductive part 14 is, for example, about several tens of micrometers. When the bar-shaped or the thread-shaped conductive part 14 is used, it is effective to provide the auxiliary contact surface 14a at a contact site between the conductive part 14 and the electrode 11; however, the configuration where the auxiliary contact surface 14a is not provided is also practicable.

It is allowable to use either the opening-type conductive part 14 or the bar-shaped or thread-shaped conductive part 14 or both of them.

The explanation will be continued with referring back to FIG. 1. An electrode provided on the right side of the steering wheel 1 is connected to an Op-Amp OP1 via a switch SW1. In the same manner, an electrode provided on the left side of the steering wheel 1 is connected to an Op-Amp OP2 via a switch SW2. The Op-Amps OP1 and OP2 are grounded as a reference. In the field of vehicles, grounding means connecting an electrode to the vehicle frame and the reference potential is the potential of the vehicle frame. This reference potential is called frame ground (FG). The right and left directions of the steering wheel 1 depend on the driver's viewpoint.

Therefore, when the right hand of the driver touches the right side of the steering wheel 1, an electrical condition is detected from the right hand of the driver, and then amplified and output by the Op-Amp OP1. In the same manner, when the left hand of the driver touches the left side of the steering wheel 1, an electrical condition is detected from the left hand of the driver, and then amplified and output by the Op-Amp OP2.

As described later, the seat-surface electrode part 20 is capacitive coupling electrodes: one electrode is connected to an Op-Amp OP3 via a switch SW3 and the other electrode is grounded. The Op-Amp OP3 is also grounded as a reference

Therefore, when the driver sits on the driver seat 2, an electrical condition is detected from the buttocks of the driver, and then amplified and output by the Op-Amp OP3.

The switch S1 is between the electrode of the steering wheel 1 and the Op-Amp OP1 and the switch S2 is between the electrode of the steering wheel 1 and the Op-Amp OP2: they allow the electrical conditions detected by the steering wheel 1 to flow to the ground. As a result, both of the inputs to the Op-Amps OP1 and OP2 are grounded and the outputs of the Op-Amps OP1 and OP2 become zero. In other words, the switches S1 and S2 operate as switching units that switch whether or not the electrical conditions of the driver are to be detected from the steering wheel 1.

The switch S3 is between the electrode of the driver seat 2 and the Op-Amp OP3: it allows the electrical condition detected by the seat-surface electrode part 20 to flow to the ground. As a result, the input to the Op-Amp OP3 is grounded and the output of the Op-Amp OP3 becomes zero. In other words, the switch S3 operates as a switching unit that switches whether or not the electrical condition of the driver is detected from the seat-surface electrode part 20.

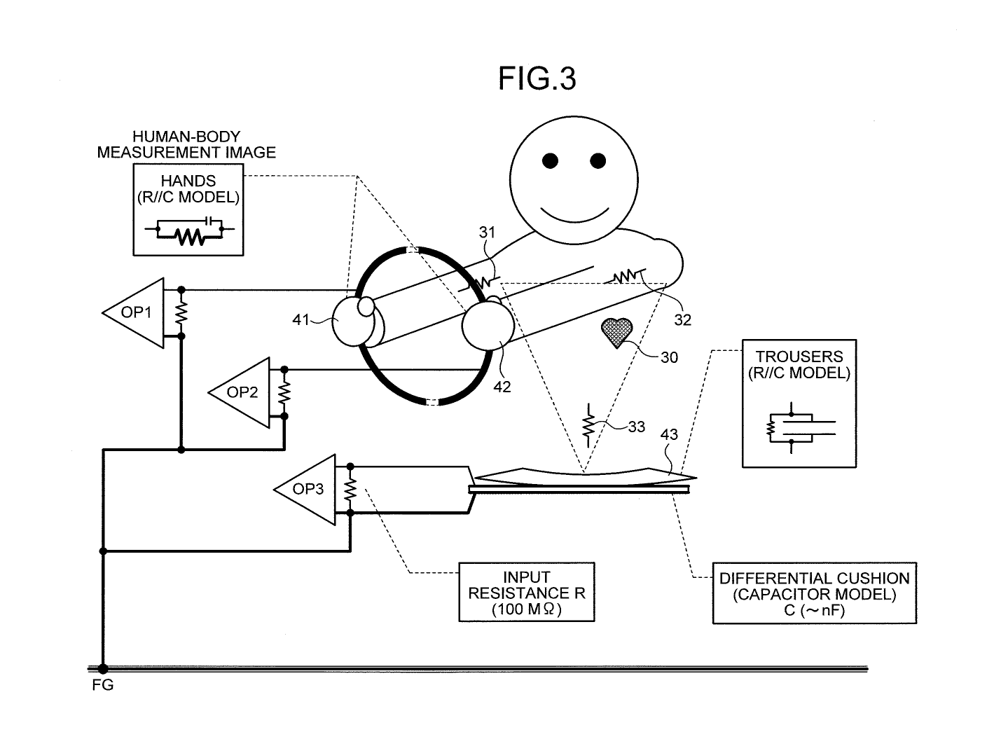

FIG. 3 is a diagram that explains detection of the electrical conditions of the driver. A situation where the driver sits on the driver seat 2 and grips the right side of the steering wheel 1 with the right hand and the left side of the steering wheel 1 with the left hand is illustrated in FIG. 3. In the situation illustrated in FIG. 3, the Op-Amp OP1 detects an electrocardiographic signal of the driver from the right hand. In the same manner, the Op-Amp OP2 detects an electrocardiographic signal of the driver from the left hand. The Op-Amp OP3 detects an electrocardiographic signal of the driver from the buttocks.

A part of the driver from a heart 30 to the arms is electrically assumed to be a resistance component. The hands of the driver are electrically assumed to be RC parallel circuits. Similarly, a part of the driver from the heart 30 to the buttocks is electrically assumed to be a resistance component. The cloths, such as trousers, are electrically assumed to be an RC parallel circuit.

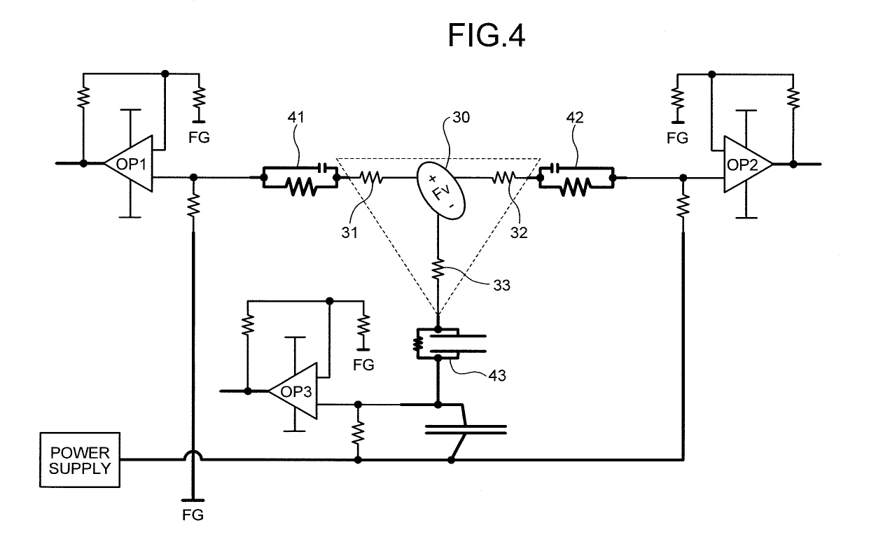

Assuming that a part of the driver from the heart 30 to the right arm is a resistance 31, the right hand is an RC parallel circuit 41, a part of the driver from the heart 30 to the left arm is a resistance 32, the left hand is an RC parallel circuit 42, a part from the heart 30 to the buttocks is a resistance 33, and the cloths are an RC parallel circuit 43, an equivalent circuit including the driver himself/herself is designed as illustrated in FIG. 4.

The cardiac action potential of the heart 30 of the driver changes periodically depending on the heart beat. The periodical change in the cardiac action potential is output as electrocardiographic signals from the Op-Amps OP1 to OP3. The Op-Amp OP1 amplifies the periodical change in the cardiac action potential that is input via the resistance 31 and the RC parallel circuit 41 and then outputs it as an electrocardiographic signal. The Op-Amp OP2 amplifies the periodical change in the cardiac action potential that is input via the resistance 32 and the RC parallel circuit 42 and then outputs it as an electrocardiographic signal. Similarly, the Op-Amp OP3 amplifies the periodical change in the cardiac action potential that is input via the resistance 33 and the RC parallel circuit 43 and then outputs it as an electrocardiographic signal. It is noted that a power supply for amplification by the Op-Amps OP1 to OP3 can be implemented by using a DC converter or the like based on a vehicle battery.

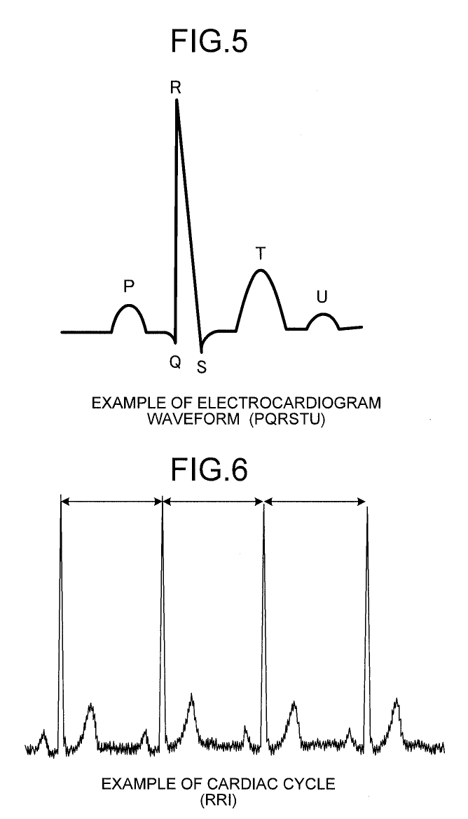

FIG. 5 is an explanatory diagram of an electrocardiogram waveform. The electrocardiogram waveform has maximum values P, R, T, and U and minimum values Q and S. Because the maximum value R is the largest among them, the intervals between the maximum values R are measured as illustrated in FIG. 6 to detect psychological conditions and physical conditions, such as a change in the arousal level of the driver.

The electrocardiogram waveform is detectable by using a single electrocardiographic signal that is output from any of the three Op-Amps OP1 to OP3. However, if a plurality of, for example, two outputs of Op-Amps are used, it is possible to reduce a noise component and increase the accuracy of the detected electrocardiogram waveform. For example, when the driver grips the steering wheel with the both hands to be able to obtain electrocardiographic signals from both of the Op-Amp OP1 and the Op-Amp OP2, then the electrocardiogram waveform is detected by using the outputs of the Op-Amp OP1 and the Op-Amp OP2. When the driver grips the steering wheel with only the right hand with the left hand being away from the steering wheel, the electrocardiogram waveform is detected by using the outputs of the Op-Amp OP1 and the Op-Amp OP3. When the driver grips the steering wheel with only the left hand with the right hand being away from the steering wheel, the electrocardiogram waveform is detected by using the outputs of the Op-Amp OP2 and the Op-Amp OP3. It is preferable that, regarding any Op-Amp whose output is unused, any of the switches SW1 to SW3 that corresponds to the unused Op-Amp is switched so that the output becomes zero.

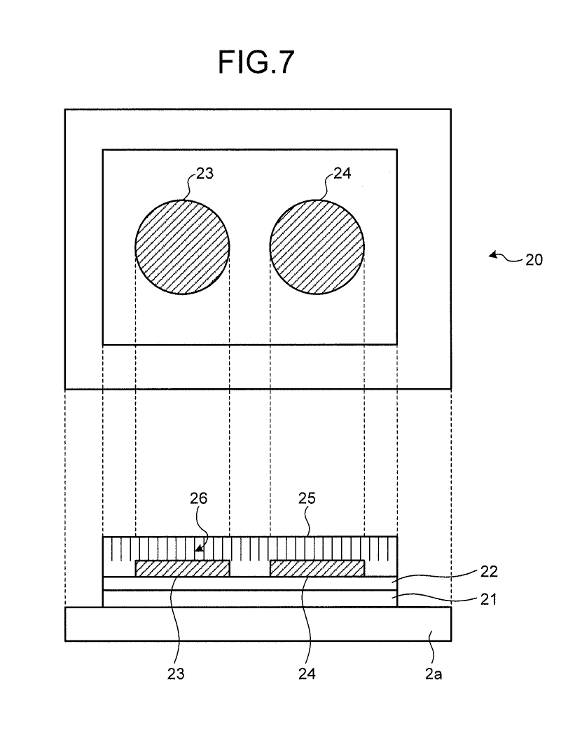

Then, the structure of the seat-surface electrode part and a modification thereof are described. FIG. 7 is a structure diagram of the seat-surface electrode part that detects electrocardiographic signals from the right buttock and the left buttock. The seat-surface electrode part 20 illustrated in FIG. 7 has a laminated structure in which a lower electrode 21, an insulating layer 22, upper electrodes 23 and 24, and a protective member 25 are formed on a seat member 2a.

The protective member 25 has conductive parts 26 in the same manner as in the protective member 12. Each of the conductive parts 26 can be provided on an inner wall of a hole having open ends or can be formed as a bar-shaped or thread-shaped conductive part. The conductive parts 26 are in contact with the upper electrodes 23 and 24. The upper electrodes 23 and 24 are electrically independent from each other and respectively detect electrical conditions from the right buttock and the left buttock of the driver.

The lower electrode 21 faces the upper electrodes 23 and 24 while sandwiching the insulating layer 22 therebetween. The lower electrode 21 is grounded. With this configuration, each of a pair of the lower electrode 21 and the upper electrode 23 and a pair of the lower electrode 21 and the upper electrode 24 operates as a capacitive coupling electrode.

The configuration of FIG. 7 indicates that two upper electrodes are provided to detect electrical conditions from the right buttock and the left buttock of the driver. However, if there is only one upper electrode, it is configured that one electrical condition is detected from the buttocks of the driver as illustrated in FIG. 1. Although the configuration of FIG. 7 indicates that the common lower electrode is used, it is allowable to provide individual lower electrodes in accordance with the two upper electrodes.

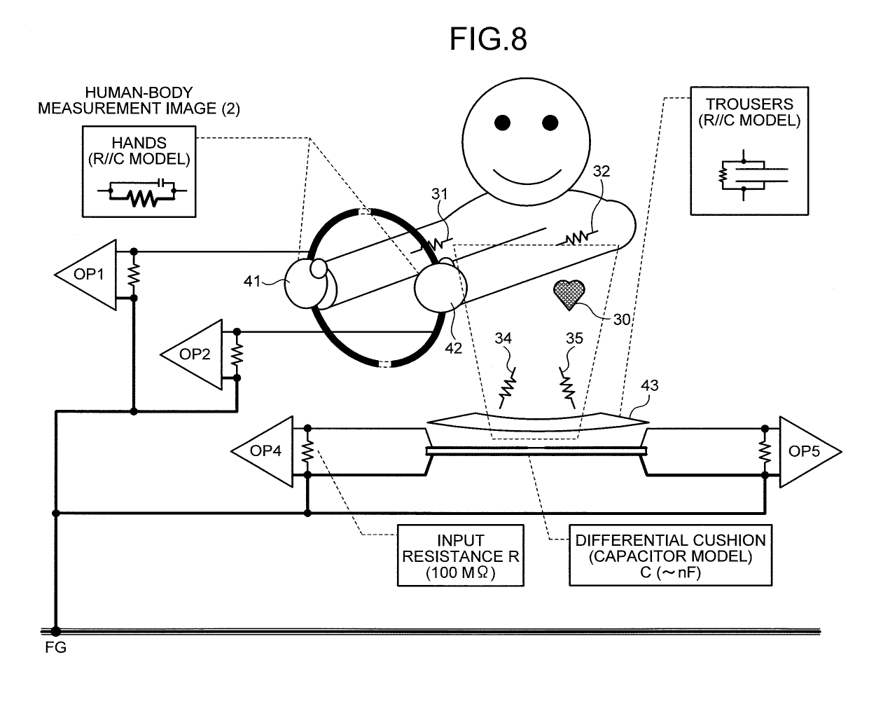

FIG. 8 is a diagram that explains a way of individually detecting electrical conditions from the right buttock and the left buttock of the driver. Similarly to FIG. 3, FIG. 8 illustrates a situation where the driver sits on the driver seat 2 and grips the right side of the steering wheel 1 with the right hand and the left side of the steering wheel 1 with the left hand. In the situation illustrated in FIG. 8, the Op-Amp OP1 detects an electrocardiographic signal of the driver from the right hand. In the same manner, the Op-Amp OP2 detects an electrocardiographic signal of the driver from the left hand. An Op-Amp OP4 detects an electrocardiographic signal of the driver from the right buttock. An Op-Amp OP5 detects an electrocardiographic signal of the driver from the left buttock.

When electrical conditions are detected from the right buttock and the left buttock of the driver, a part of the driver from the heart 30 to the right buttock and a part of the driver from the heart 30 to the left buttock are assumed to be individual resistance components.

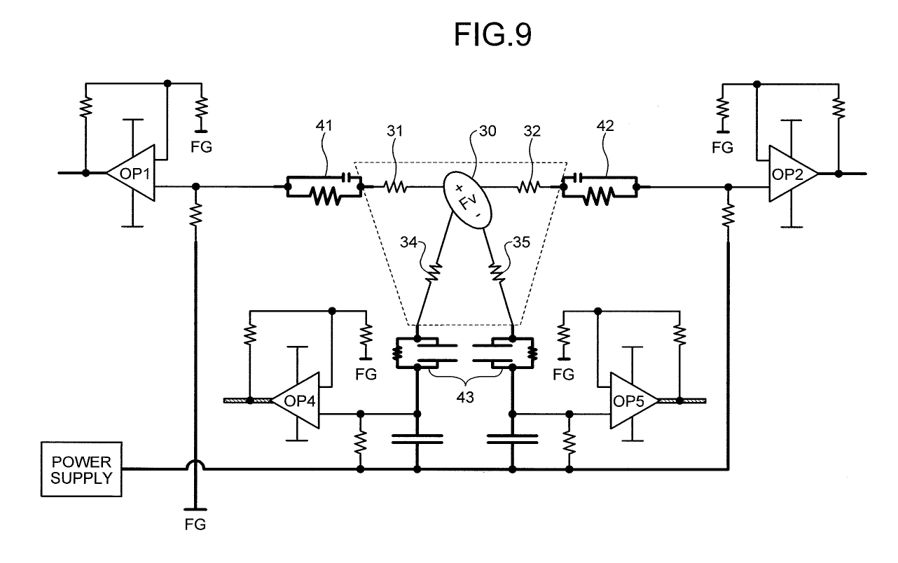

If the part of the driver from the heart 30 to the right buttock is a resistance 34 and the part of the driver from the heart 30 to the left buttock is a resistance 35, then an equivalent circuit including the driver himself/herself is designed as illustrated FIG. 9.

In FIG. 9, the Op-Amp OP1 and the Op-Amp OP2 output electrocardiographic signals in the same manner as in FIG. 3. The Op-Amp OP4 amplifies the periodical change in the cardiac action potential that is input via the resistance 34 and the RC parallel circuit 43 and then outputs it as an electrocardiographic signal. The Op-Amp OP5 amplifies the periodical change in the cardiac action potential that is input via the resistance 35 and the RC parallel circuit 43 and then outputs it as an electrocardiographic signal.

The electrocardiogram waveform is detectable by using a single electrocardiographic signal that is output from any of the four Op-Amps OP1, OP2, OP4, and OP5. Moreover, it is allowable to select arbitrary two from the electrocardiographic signals that are output from any of the four Op-Amps OP1, OP2, OP4, and OP5 and use them for detection of the electrocardiogram waveform. For example, even if it is impossible to acquire any electrocardiographic signal from the hands of the driver, i.e., the outputs of the Op-Amps OP1 and OP2 are unavailable; it is possible to detect an accurate electrocardiogram waveform by using the outputs of the Op-Amps OP3 and OP4.

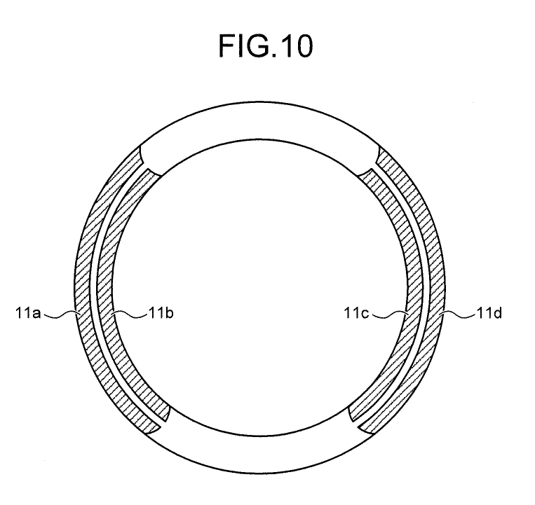

Then, a modification of the electrodes provided on the steering wheel 1 will be explained. FIG. 10 is a diagram that explains a configuration in which electrodes are provided on the outside and the inside of the steering wheel. In the configuration illustrated in FIG. 10, an electrode 11a is provided on the left-side outer circumference of the steering wheel 1 and an electrode 11b is provided on the left-side inner circumference of the steering wheel 1. In the same manner, an electrode 11c is provided on the right-side outer circumference of the steering wheel 1 and an electrode 11d is provided on the right-side inner circumference of the steering wheel 1.

The electrode 11a is electrically independent from the electrode 11b. The electrode 11c is electrically independent from the electrode 11d. Therefore, the electrical conditions of the driver are detectable from the inner circumference and the outer circumference of the left side of the steering wheel 1 and the inner circumference and the outer circumference of the right side of the steering wheel 1.

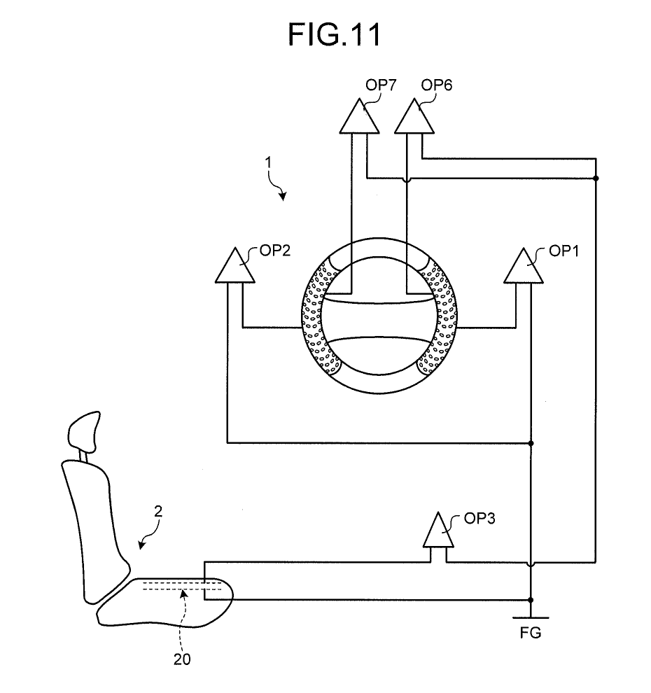

FIG. 11 is a diagram of a configuration in which electrical conditions are detected from the outside and the inside of the steering wheel 1. As illustrated in FIG. 11, the electrode 11a that is on the outer circumference of the left side of the steering wheel 1 is connected to the Op-Amp OP2. The electrode 11b that is on the inner circumference of the left side of the steering wheel 1 is connected to an Op-Amp OP7. In the same manner, the electrode 11d that is on the outer circumference of the right side of the steering wheel 1 is connected to the Op-Amp OP1. The electrode 11c that is provided on the right-side inner circumference of the steering wheel 1 is connected to an Op-Amp OP6. The Op-Amps OP1, OP2, OP6, and OP7 are grounded as a reference. Although not illustrated in FIG. 11, in the same manner as in FIG. 1, a switch is on each channel that is used to input an electrical condition of the driver to any of the Op-Amps OP1 to OP3, OP6, and OP7 so that it is possible to switch the output of any Op-Amp to zero. Because the other configuration and operations are the same as those of FIG. 1, the same explanation is not repeated.

If, as illustrated in FIG. 11, the electrode of the steering wheel 1 is separated into two, one being on the inside and the other being on the outside, even if the driver grips the steering wheel 1 with a single hand, it is possible to detect electrical conditions of the driver by using two systems from the steering wheel 1. Because two electrocardiographic signals acquired from the two systems are used, the electrocardiogram waveform is detected more accurately than the electrocardiogram waveform detected by using one electrocardiographic signal. Moreover, it is possible to select arbitrary two from the outputs of the Op-Amps OP1 to OP3, OP6, and OP7 and detect the electrocardiogram waveform.

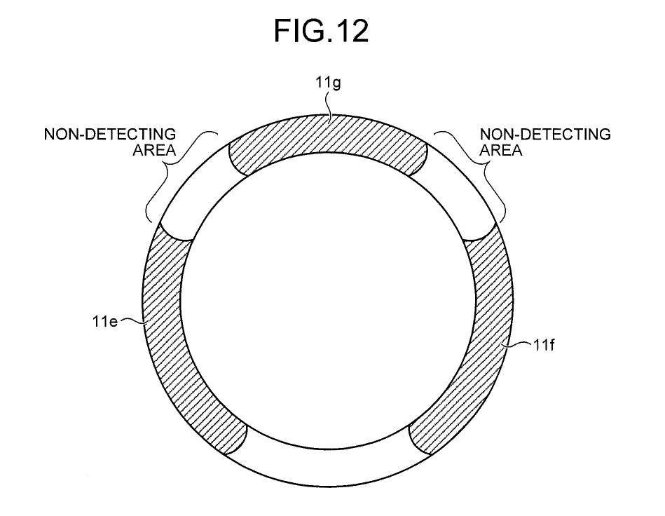

FIG. 12 is a diagram that explains a configuration in which electrodes are provided on not only the right side and the left side of the steering wheel 1 but also the upper side of the steering wheel 1. In the configuration illustrated in FIG. 12, an electrode 11e is provided on the left side of the steering wheel 1, an electrode 11f is provided on the right side of the steering wheel 1, and an electrode 11g is provided on the upper side of the steering wheel 1.

A driver would manipulate the steering wheel 1 with one hand while laying the one hand on the upper side of the steering wheel 1. Because, as illustrated in FIG. 12, the electrode 11g is provided on the upper side of the steering wheel 1, it is possible to detect an electrical condition of the driver from the hand touching the upper side of the steering wheel 1. When the other hand touches the electrode 11e or the electrode 11f, is possible to acquire electrocardiographic signals from the both hands and use them for detection of the electrocardiogram waveform.

Non-detecting areas are provided between the electrode 11e and the electrode 11g and between the electrode 11f and the electrode 11g so that the electrodes are separated from each other. It is preferable to set the width of the non-detecting areas to a value wider than the width of the palms. By setting the width of the non-detecting areas to a value wider than the palms, a situation is prevented that either hand of the driver touches a plurality of electrodes and the electrodes detect electrical conditions from the same part of the driver.

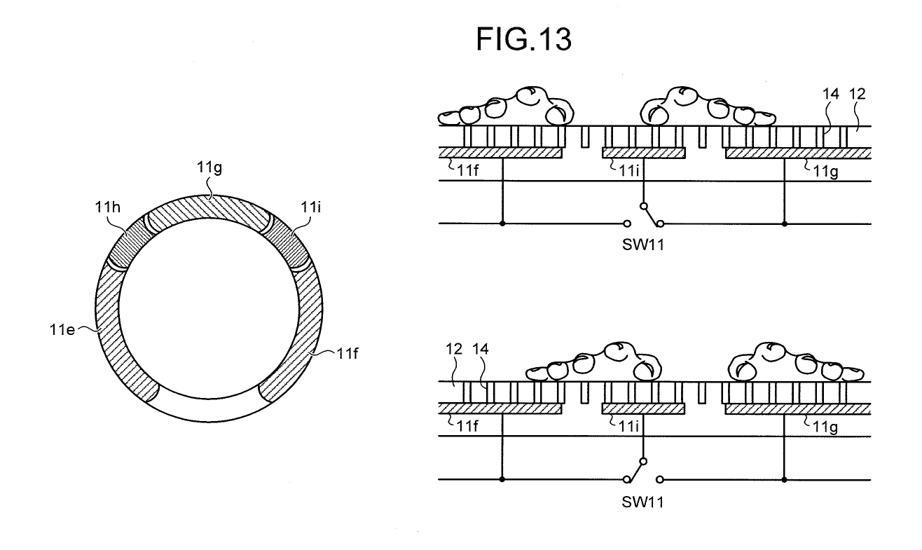

FIG. 13 is a diagram that explains a configuration in which auxiliary electrodes are provided on the steering wheel 1. In the configuration illustrated in FIG. 13, the electrode 11e is provided on the left side of the steering wheel 1, the electrode 11f is provided on the right side of the steering wheel 1, and the electrode 11g is provided on the upper side of the steering wheel 1. Moreover, an auxiliary electrode 11h is provided between the electrode 11e and the electrode 11g, and an auxiliary electrode 11i is provided between the electrode 11f and the electrode 11g.

The auxiliary electrode 11h is an electrode whose modes are switchable so that it operates as either the electrode 11e or the electrode 11g. The auxiliary electrode 11i is an electrode whose modes are switchable so that it operates as either the electrode 11g or the electrode 11f.

An example of switching of the auxiliary electrode 11i is illustrated in FIG. 13. When the left hand of the driver is over both the auxiliary electrode 11i and the electrode 11g, the auxiliary electrode 11i operates as the electrode 11g. In contrast, when the left hand of the driver is over both the auxiliary electrode 11i and the electrode 11f, the auxiliary electrode 11i operates as the electrode 11f. The modes of the auxiliary electrode 11i that operates as either electrode are switched by a switch SW11.

The hands of the driver touch the electrodes and the auxiliary electrodes via the conductive parts 14 that go through the protective member 12. Some of the conductive parts 14 formed on the protective member 12 are connected to the electrodes 11e, 11f, and 11g. In the same manner, some of the conductive parts 14 are connected to the auxiliary electrodes 11h and 11i. The conductive parts 14 can include a conductive part that is connected to neither the electrodes nor the auxiliary electrodes.

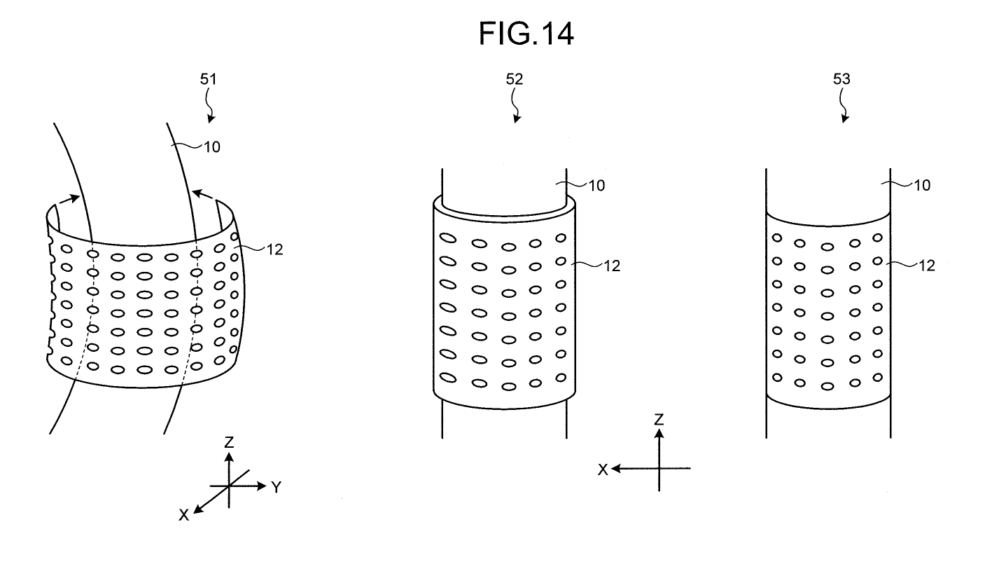

FIG. 14 is a diagram that explains the shape of the holes 13 formed on the protective member 12. When the protective member 12 having holes evenly formed thereon is wound onto the steering-wheel structure 10, as illustrated in a steering-wheel perspective view 51, the outside protective member of the steering wheel are stretched more widely than the inside protective member of the steering wheel. Therefore, if the elliptical holes 13 are formed in such a manner that the minor axis is aligned with the circumferential direction of the steering wheel 1, the ratio between the major axis and the minor axis of the holes on the outer circumference is larger than the ratio between the major axis and the minor axis of the holes on the inner circumference.

Because manipulation of the steering wheel 1 includes many actions of sliding along the outer circumference and the outer circumference is likely to be worn away, an increase in the ratio between the major axis and the minor axis of the holes on the outer circumference is effective for improvement of the durability.

When comparing the front side of the steering wheel or the driver side with the rear side of the steering wheel or the vehicle side, abrasion due to the manipulation is likely to occur on the front side. In FIG. 14, the positive X direction corresponds to the direction toward the front side; the negative X direction corresponds to the direction toward the rear side.

To increase the durability on the front side of the steering wheel 1, it is preferable to put the center part of the protective member 12 on the front side of the steering wheel 1 and sew it on the rear side of the steering wheel 1. When the center part of the protective member 12 is put on the front side of the steering wheel 1 and then sewn it on the rear side of the steering wheel 1, as illustrated in a steering-wheel side view 52, the ratio between the major axis and the minor axis of the holes is increased on the front side or the positive side in the X direction and the difference between the major axis and the minor axis of the holes is increased on the rear side or the negative side in the X direction.

It is noted that the ratio between the major axis and the minor axis of the holes near the steering-wheel structure 10 is less than the ratio between the major axis and the minor axis of the holes on the surface of the protective member 12. A steering-wheel structure surface view 53 illustrates the holes near the steering-wheel structure 10. As illustrated, the shape of the holes on the surface of the protective member 12 is different from the shape of the holes on the side of the steering-wheel structure 10 because of the difference between the distances away from the center of the steering-wheel structure 10.

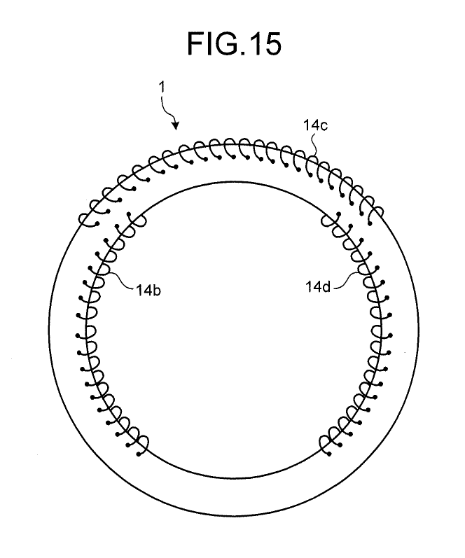

FIG. 15 is a diagram that explains an example in which the protective member 12 is sewn with conducting thread. The protective member 12 is sewn with conducting thread and the sewing thread operates as the conductive parts 14 that go through the protective member 12 and come into contact with the electrodes. In the example illustrated in FIG. 15, the stitches on the left side of the steering wheel 1 form conductive parts 14b. The stitches on the upper side of the steering wheel 1 form conductive parts 14c. The stitches on the right side of the steering wheel 1 form conductive parts 14d.

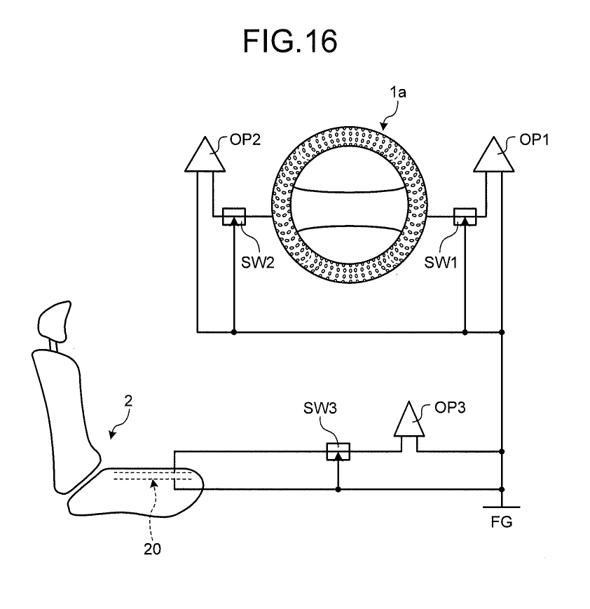

FIG. 16 is a diagram that explains a configuration in which the entire steering wheel is covered with the protective member. A steering wheel 1a illustrated in FIG. 16 has a steering wheel part entirely covered with the protective member 12. The protective member 12 has holes evenly formed thereon: each hole has a conductive part therein. In contrast, electrodes are arranged on some parts of the steering wheel 1a under the protective member 12. Therefore, only some conductive parts being in contact with the electrodes transfer electrical conditions of the driver to the electrodes.

In the same manner as in the steering wheel 1 illustrated in FIG. 1, the steering wheel 1a detects the electrical conditions of the driver from the right side and the left side of the steering wheel 1a. Because the layout of the electrodes is hidden behind the protective member 12 that covers the entire wheel part of the steering wheel 1a, the steering wheel 1a can take any design without affecting the layout of the electrodes.

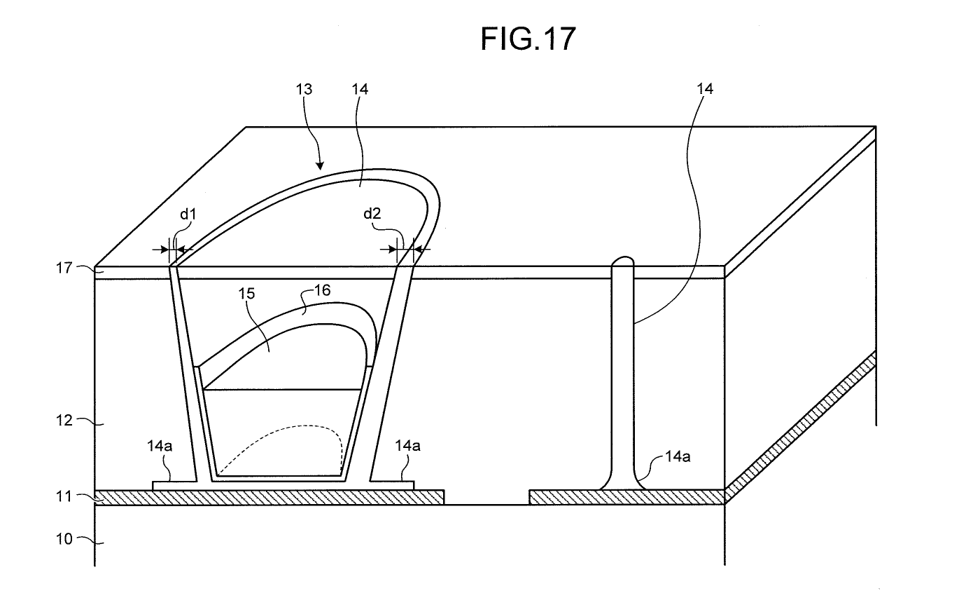

FIG. 17 is a diagram that explains a configuration in which a conductive layer 17 is further formed on the surface of the protective member 12. In the configuration illustrated in FIG. 17, the conductive layer 17 is formed on the surface of the protective member illustrated in FIG. 2. Because the other configuration is the same as that of FIG. 2, the same description is not repeated. The conductive layer 17 formed on the surface of the protective member 12 helps the driver to touch the conductive parts 14 with his/her body, which helps detection of an electrical condition.

As mentioned above, because it is configured to have conductive parts that pass through a protective member and come into electrical contact with electrodes that are formed under the protective member, it is possible to detect the electrical condition of an operator while increasing the durability and preventing a loss in the degree of freedom in design by easing the restrictions on materials and shape.

The present embodiment is merely an example and the disclosed technology can be implemented as an appropriate modification. Although, for example, in the present embodiment, an example of the configuration is described in which electrodes are provided on the right side, the left side, and the upper side of the vehicle, it is allowable to, for example, provide an electrode on the lower side of the steering wheel.

Although, in the present embodiment, two electrodes are provided on the right side and the left side of the surface of the driver seat, respectively, an arbitrary number of electrodes can be provided in an arbitrary layout, for example, a layout in which an electrode is separated into a front part and a rear part. It is possible to provide an electrode on a backseat part or a headrest part of the driver seat.

Although, in the present embodiment, an electrical condition of the driver of the vehicle is detected, the technology is applicable for detection of an electrical condition of an operator of an arbitrary device. Moreover, an electrical condition of the operator is detectable from not only the steering wheel but also any steering tool, such as a lever-shaped steering tool.

As described above, according to an aspect of the present invention, the electric condition of an operator can be detected without losing the degree of freedom in design by increasing the durability and easing the restrictions on materials and shapes.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the spirit and scope of the invention.

What is claimed is:

1. An operator condition detecting device comprising:
an electrode part that detects an electrical condition of an operator of an apparatus;
a protective layer that covers the electrode part;
a plurality of holes that passes through the protective layer; and
conductive parts that are formed on inner walls of the holes to be in contact with the electrode part.

2. The operator condition detecting device according to claim 1, wherein an electrode-side open end of each the hole is smaller than a protective-layer-surface-side open end of the hole.

3. The operator condition detecting device according to claim 1, wherein an electrode-side part of each the hole is filled with a protective agent.

4. The operator condition detecting device according to claim 1, wherein the conductive parts are formed by sewing the protective layer with conducting thread.

5. The operator condition detecting device according to claim 1, wherein electrical conditions are detected from a plurality of sites of the operator and an electrocardiogram is calculated from the detected electrical conditions.

6. The operator condition detecting device according to claim 1, wherein
the apparatus that is steered by the operator is a vehicle, and
the electrode part, the protective layer, and the conductive parts are provided in a steering wheel and/or a seat of the vehicle.

7. The operator condition detecting device according to claim 6, wherein the electrode part includes a first electrode that detects the electrical condition from one hand of the operator and a second electrode that detects the electrical condition from another hand of the operator.

8. The operator condition detecting device according to claim 7, wherein
the electrode part further includes an auxiliary electrode between the first electrode and the second electrode, and
the auxiliary electrode is switched to and used as either the first electrode or the second electrode.

9. The operator condition detecting device according to claim 6, wherein the electrode part is provided on an outside of the steering wheel and an inside of the steering wheel.

10. The operator condition detecting device according to claim 6, wherein an operator-side layout of the conductive parts of the steering wheel is denser than a vehicle-side layout of the conductive parts of the steering wheel.

11. The operator condition detecting device according to claim 6, wherein
the conductive parts are formed on the inner walls of the holes having an elliptical shape, and
a major axis of the ellipse is aligned with a radial direction of the steering wheel.

12. A steering wheel comprising:
an electrode part that detects an electrical condition of an operator who conducts a driving action;
a protective layer that covers the electrode part; and
conductive parts that pass through the protective layer to
be in contact with the electrode part.
